(19) United States
(12) Patent Application Publication
(10) Pub. No.: US 2019/0265084 A1
(43) Pub. Date: Aug. 29, 2019

(54) FLUID DETECTION FLANGE FOR A PRODUCT TRANSFER VERIFICATION SYSTEM

(71) Applicant: SCULLY SIGNAL COMPANY, Wilmington, MA (US)

(72) Inventor: William A. Breckenridge, Alberta (CA)

(73) Assignee: SCULLY SIGNAL COMPANY, Wilmington, MA (US)

(21) Appl. No.: 16/340,837

(22) PCT Filed: Oct. 11, 2017
(86) PCT No.: PCT/US2017/056034

§ 371 Date: Apr. 10, 2019

Related U.S. Application Data
(60) Provisional application No. 62/406,628, filed on Oct. 11, 2016.

(51) Int. Cl. G01D 11/24; G01N 33/28; H04Q 9/00; H04L 12/40

(52) U.S. Cl. CPC G01D 11/245; H04L 12/40; H04Q 9/00; G01N 33/28

(57) ABSTRACT

A fluid detection flange for a product transfer verification system includes a coupling frame having an opening defining a fluid path configured to be fluidly coupled with a fluid source. A sensor in the flange measures at least one characteristic of a fluid in the opening and the sensed characteristic is used to determine the type of fluid pending for transfer. In operation, if the pending fluid is the same type as is meant to be placed in a destination container then the transfer is allowed.

### BACKGROUND

This application is claiming priority to U.S. provisional patent application Ser. No. 62/406,628 entitled “Product Transfer Verification System,” filed Oct. 11, 2016, the entire contents of which is incorporated by reference herein for all purposes.

### BACKGROUND

As is known, there are many different types of fuel products used for various purposes including aviation, automotive, trucking, etc. These fuels may oftentimes be distributed from the same points and tanker trucks may be carrying different types or grades of fuel at the same time. Of course, each fuel is kept in its own respective compartment of the tanker as mixing of different fuels can be dangerous.

There are many opportunities where contamination of fuels may occur. These include the time when a tanker truck is being loaded—the correct fuel should be placed in the correct compartment and when the delivery is being made, for example, when automotive fuel is being delivered to a gas station. It is imperative that the correct fuel be placed in the correct storage tank as this impacts inventory, billing and safety systems.

Systems are known for trying to prevent the cross-contamination of fuels either in transport vehicles or upon delivery. What is needed, however, is a system that prevents the contamination with a minimum of user input especially where operator error may contribute to contamination.

### SUMMARY

In one aspect of the present disclosure, a coupling flange comprises a coupling frame having an opening defining a fluid path configured to be fluidly coupled with a fluid source; at least one sensor having a sensing portion configured to measure at least one characteristic of a fluid, the sensor provided in the coupling frame such that the sensing portion is located in the fluid path; a signal bus, provided in the coupling frame, coupled to the at least one sensor; and a transmitter module, coupled to the signal bus, configured to transmit sensor data received from the at least one sensor.

In another aspect of the present disclosure, a method of controlling a pending transfer of a fluid product to a receiving compartment comprises measuring at least one characteristic of the pending fluid product; identifying, as a function of the at least one measured characteristic, a type of the pending fluid product; determining if the identified fluid type corresponds to the receiving compartment; and preventing the pending transfer if the identified fluid type does not correspond to the receiving compartment.

Measuring the at least one characteristic of the pending fluid product comprises contacting, with a sensor probe, the fluid product in a conduit.

### BRIEF DESCRIPTION OF THE DRAWINGS

Various implementations of at least one aspect of the present disclosure are discussed below with reference to the accompanying figures. It will be appreciated that for simplicity and clarity of illustration, elements shown in the drawings have not necessarily been drawn accurately or to scale. Further, where considered appropriate, reference numerals may be repeated among the drawings to indicate corresponding or analogous elements. For purposes of clarity, not every component may be labeled in every drawing. The figures are provided for the purposes of illustration and explanation and are not intended as a definition of any limits of aspects of the present disclosure. In the figures:

FIG. 1 is a Venn Diagram representing the conditions under which loading or unloading of product is permitted;

FIG. 2 represents a truck/trailer truck 200 and a trailer 204 provided with interconnected components to facilitate the correct loading/unloading of product;

FIG. 3 is block diagram of a Trailer-to-Truck Connect Assembly (TTCA);

FIG. 4 is a block diagram of a power management controller (PMC);

FIG. 5 is a block diagram of a peripheral controller;

FIG. 6 is display controller of a product transfer verification system (PTVS) module;

FIG. 7 is a block diagram of an overfill detection system coupled to a valve controller;

FIG. 8 is a block diagram of a set of belly valves coupled through a device hub interface where each probe flange includes a respective sensor PCB assembly;

FIG. 9 is a block diagram of a sensor PCB assembly;

FIG. 10 is a representation of the system when loading product into the compartments of the trailer;

FIG. 11 is a magnified view of a portion of a loading terminal;

FIG. 12 is a representation of the system when unloading product into the compartments of a retail location;

FIG. 13 is a magnified view of a portion of the receiving retail location;

FIG. 14 is a schematic of a probe flange; and

FIG. 15 is a representation of an implementation of the probe flange of FIG. 14.

### DETAILED DESCRIPTION

This application is claiming priority to U.S. provisional patent application Ser. No. 62/406,628 entitled “Product Transfer Verification System,” filed Oct. 11, 2016, the entire contents of which is incorporated by reference herein for all purposes.

In the following detailed description, numerous specific details are set forth in order to provide a thorough understanding of the aspects of the present disclosure. It will be understood by those of ordinary skill in the art that these may be practiced without some of these specific details. In other instances, well-known methods, procedures, components and structures may not have been described in detail so as not to obscure the aspects of the present disclosure.

Prior to explaining at least one implementation of at least one aspect of the present disclosure in detail, it is to be understood that aspects of the present disclosure are not limited in their application to the details of construction and the arrangement of the components set forth in the following description or illustrated in the drawings. Other implementations are possible. Also, it is to be understood that the phraseology and terminology employed herein are for the purpose of description only and should not be regarded as limiting.

It is appreciated that certain features, which are, for clarity, described in the context of separate implementations, may also be provided in combination in a single implementation. Conversely, various features, which are, for brevity, described in the context of a single implementation, may also be provided separately or in any suitable sub-combination.

Generally, one advantage of the present system is to mitigate the risk to carriers when a driver unloads a petroleum product into the incorrect storage tank (cross drop or cross-over). The annual cost to the carrier for cross drop events is estimated to range from $1,000 to $1M. The exact frequency of these cross drop events is unknown, however, the petroleum trucking industry is seeking new technology to eliminate this hazard. The present system mitigates most of the cross drop scenarios on the delivery or unloading side of the petroleum transport equation.

In one aspect of the system, product and process information is displayed automatically, for convenience of the operator, as well as increasing accuracy of the delivery process, to serve as a visual confirmation during the fuel delivery process. In another aspect, cross-over prevention is provided by synthesizing information from three main areas: 1) product, 2) storage tank and 3) inventory, and distilling this information to determine a permit or non-permit condition, i.e., authorization to load or unload fuel.

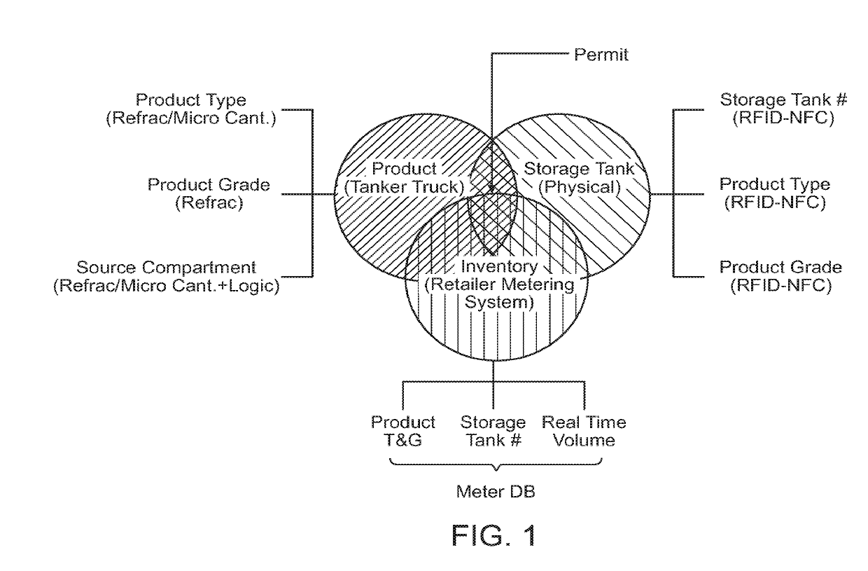

Referring to FIG. 1, loading or unloading is permitted when the following conditions exist based on inputs/objects: the product on the tanker truck has been confirmed, e.g., correct product type, correct product grade and in the correct compartment. The correct storage tank has to be identified and the product type and grade for the identified tank have to be confirmed and be the same as the tank from/to which product is to be transferred. Lastly, the inventory system has to be correct and updated. When all three sides have been confirmed and coordinated, i.e., where they intersect in the Venn Diagram of FIG. 1, then the transfer will be permitted.

By way of some background, it is noted that the commercial truck carrier assumes the risk and costs for cross drops. The carrier alone is tasked with ensuring that the right product is dispensed into the right drop tube, regardless of whether this occurs during a terminal to tanker or tanker to retailer product transfer.

Petroleum product, i.e., fuel, loading and unloading processes are manual and prone to human errors. Normal decision making is influenced by human conditions (distraction, boredom, illness), which can all contribute as factors to a cross drop occurrence. Currently, the carrier is equipped with informal processes (procedural, equipment, etc.) to mitigate this liability. Errors are a reality and the associated costs (potentially significant) are a part of doing business in the petroleum transport market sector.

Another pinch-point for the carrier is the relatively complex nature of the petroleum transport driver's functions and responsibilities, when compared to other transport sectors. The petroleum tanker driver is required to not only couple and haul the trailer but is also required to perform a series of mechanical tasks, make load/unload decisions, initiate and validate transactions, and perform arithmetic calculations without recourse to a calculator as he is busy moving hoses and overseeing the fuel transfer process.

In one aspect of the present system, a tanker truck based electronic system is provided to prevent or inhibit the incorrect loading and unloading of refined fuel products. The system couples a combination of wireless dispatch, terminal and retail information, with product measurements, and provides the operator/driver with an automated permit/non-permit status. The driver will still retain ultimate load/unload authorization albeit through a simple acknowledgement.

### Pre-Loading

Currently, when a fuel carrier transport trailer arrives at a fuel terminal, the assumption is that the individual compartments to be filled are empty. This is not always the case. Occasionally, these compartments will have retained product from a previous delivery because the foot valves (individual compartment valves) have closed before all of the product has been dispensed. This can occur for a couple of reasons: (1) the trailer system air pressure has dropped below a threshold value and the foot valve can no longer remain actuated and the valve has closed unbeknownst to the driver; (2) the previous product delivery volume for a given compartment exceeded the customer's tank ullage and therefore could not be fully unloaded. Another possibility is the scenario where a driver couples to a trailer with retained product that was transported by another driver. In this case, there is a reasonable margin for error as the driver is forced to rely on another driver's actions and on the rigor of the company's operating procedures and documentation.

Each of these outcomes is less than ideal and presents a crossover risk. To mitigate this risk, the driver should, at all times, be made aware of product type & grade and volume for each trailer compartment. These are key trailer payload metrics and are very important throughout the petroleum transfer process. If captured, logged and disseminated consistently and reliably during the loading process, the risk of loading on retains will be significantly reduced.

Other pre-load tasks require the driver to interpret (read) a printed dispatch ticket and manually map the compartments and set the product grade indicators (PGI) for each. If the driver is having a bad day and incorrectly maps and or sets the PGIs, then there is a risk that a crossover may occur. In fact, there are a few things that can go wrong here: (1) the driver misinterprets the ticket and loads the wrong product(s); (2) the driver is at the wrong terminal and there is no allocation; or (3) the driver forgets to set the PGIs and the compartment contents are improperly indicated. These errors can be eliminated by capturing and comparing product information from dispatch, tanker trailer and the terminal per the present system.

### System Pre-loading Summary:

Onboard (tanker trailer mounted) device that is able to measure and determine liquid refined fuel product type and grade or has the ability to differentiate between product and grades on a reference measurement basis.

Device to determine product volume for each compartment at any given moment in time.

Device to acquire, store and compare load instructions (product & volume).

Based on the foregoing information, the system will determine the compartment loading scheme (product to compartment mapping) and set the product identification for each compartment and forward this information to a local onboard and remote output.

### Loading

Currently, per known systems, assuming the compartment(s) are verifiably empty, then the driver can begin the loading process. This requires the driver to make basic yet impactful decisions and perform a series of mechanical connections. For instance, the driver must decide which loading arm dispenses which product, and then connect the loading arm to the correct tanker API valve. Once the loading arm(s) are connected to the tanker API valve(s), the driver swipes into (credential validation) the terminal controller and enters the set point(s) instructions for metered loading. The driver may relocate the loading arm to another API once the metered load is finished and commence another loading sequence through the terminal controller.

There are, however, at least two problems with how this requirement is currently fulfilled: (1) there is nothing preventing the driver from cross connecting a loading arm to the incorrect API valve, e.g., gas to diesel or Unleaded to Super; or (2) the terminal controller does not completely validate the load instructions with the carrier dispatch request or the tanker trailer which adds even more uncertainty.

Once the product is flowing from the loading arm, through the API valve and into the compartment, the driver assumes that the selected product (via the terminal controller) is actually the product being loaded. For 99.99% of the time this may be true, however, in the 0.01% of the time that it is not true there is a risk that a cross load could occur.

### Advantages:

Aspects of the present system provide for oversight of the loading process (trailer perspective) and look for proper mechanical, electrical and logical conditions in order to issue a permit condition. These conditions may be acquired locally (on the trailer) or remotely from the terminal controller/terminal equipment (loading arms).

The loading arm—API valve connection is “keyed” (mechanically, electrically, logically) in such a way that only a product type and grade match, as per the pre-load mapping, will unlock the main valve and the API valve and permit loading.

Notwithstanding existing terminal loading arm product identification, the present system measures and uniquely identifies, and confirms, the product type and grade being loaded at the earliest point in time after the fluid flows into the API. If a product mismatch is detected during loading, the system will no longer assert a “permit” condition, which in turn will disable the terminal automation system (rack controller), and product will stop flowing. Any incorrect fuel on board would have to be disposed of at a fuel recovery facility.

The product flow rate into the API will be measured and totalized to calculate accumulated volume (corrected) for each compartment.

A storage and retrieval scheme within the system captures the loading event for use internally (product reference, payload) and externally to reconcile load instructions.

### Unloading

The unloading process can involve more than one delivery location, e.g., one loaded tanker truck making multiple unloads at multiple locations, however, the exemplary discussion herein will pertain to a single location only.

In the currently known scenario, the petroleum transport arrives at the retail delivery location, e.g., a gas station, to unload the compartment contents based on the dispatch ticket instructions. The delivery almost always involves the unloading of multiple products/compartments which can create confusion for the driver. To eliminate confusion and imminent mistakes, there becomes a need to physically align the compartment to storage tank hose connection and confirm/verify the contents flowing through this connection prior to issuing a permit condition. Another consideration here is the storage tank contents and ullage. The tanks are identified by a color coded lid, e.g., White=Unleaded, Blue=Super, Yellow=Diesel, and some sort of ID tag on the filling collar, e.g., a tag with an electronic identifier (ESN) provided on, or in, it. To determine the tank ullage, as is known, the driver will stick the tank and make an eyeball reading of the height of the fluid in the tank based on the stick reading. The height is used to calculate current fluid volume which is then subtracted from the maximum fill volume to determine available tank ullage.

The problem common to the aforementioned unloading series is that these tasks are manually performed and, therefore, disconnected from one another. At no time during unloading (before or after) does the driver verify storage tank contents or ullage through the retailer Automatic Tank Gauging (ATG) system. These tasks pose a verification and acknowledgement breach between the tanker and the retailer that can lead to a product cross drop.

Functions of the system with respect to unloading:

The system is aware of geographic location (physical) and able to confirm target delivery location against dispatch ticket coordinates.

The system aligns and verifies proper tanker API valve to storage tank hose connections. This may include similar “keying” arrangements as mentioned above with respect to the loading side.

Product type and grade measurements occur prior to the fluid entering the API valve for verification. If a match (product type/grade call) is found, then the main valve and the API valve will be unlocked to permit unloading.

The system establishes a communications link between then tanker and the ATG system. The ATG system's inventory information, e.g., tank ID, contents, ullage, etc., will be gathered and used by the present system as part of permit decision making.

The synthesized information from the dispatch ticket, tanker trailer and ATG information will be stored locally and will be able to be recalled as unique items (internal calculations or external QA/QC) or as a complete event.

A communication link, either separate or using the link above, is established with the tractor fleet logistics On-Board Computer (OBC) to enable multi-system collaboration and data exchange (tanker and carrier).

Product metering occurs as compartment contents are unloaded. Advantageously, a reliable reasonably accurate measurement (+/−10 gal) to verify unloaded volume can be then be provided.

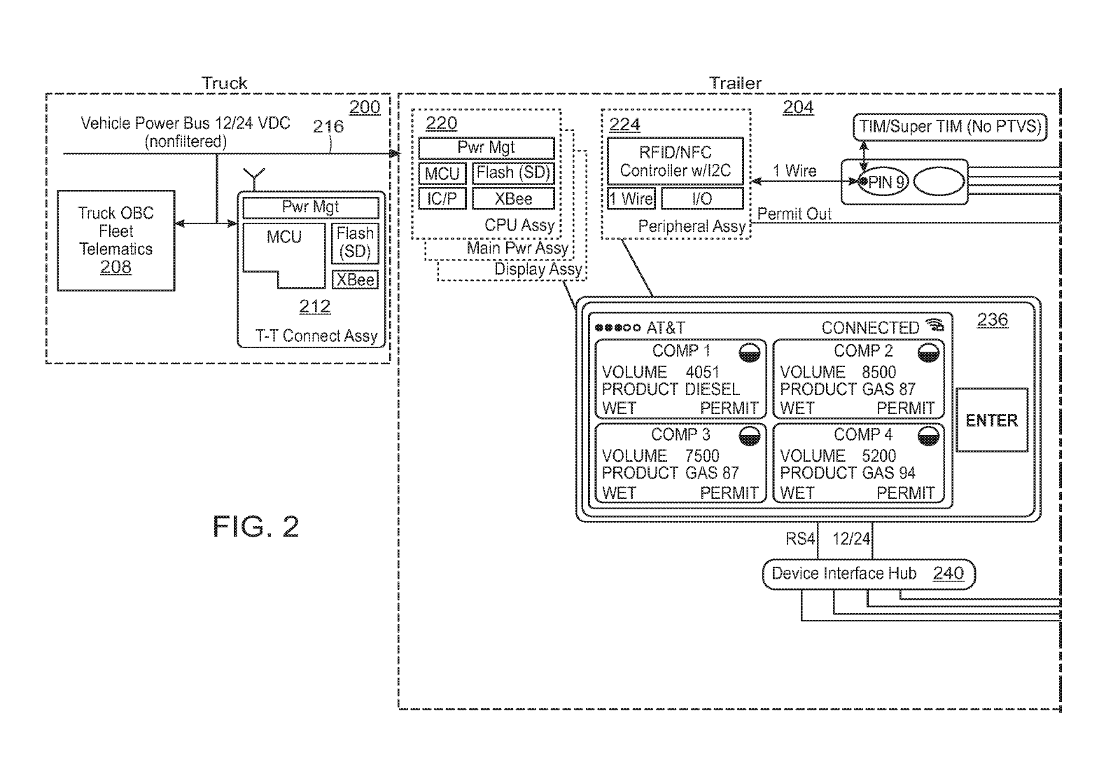

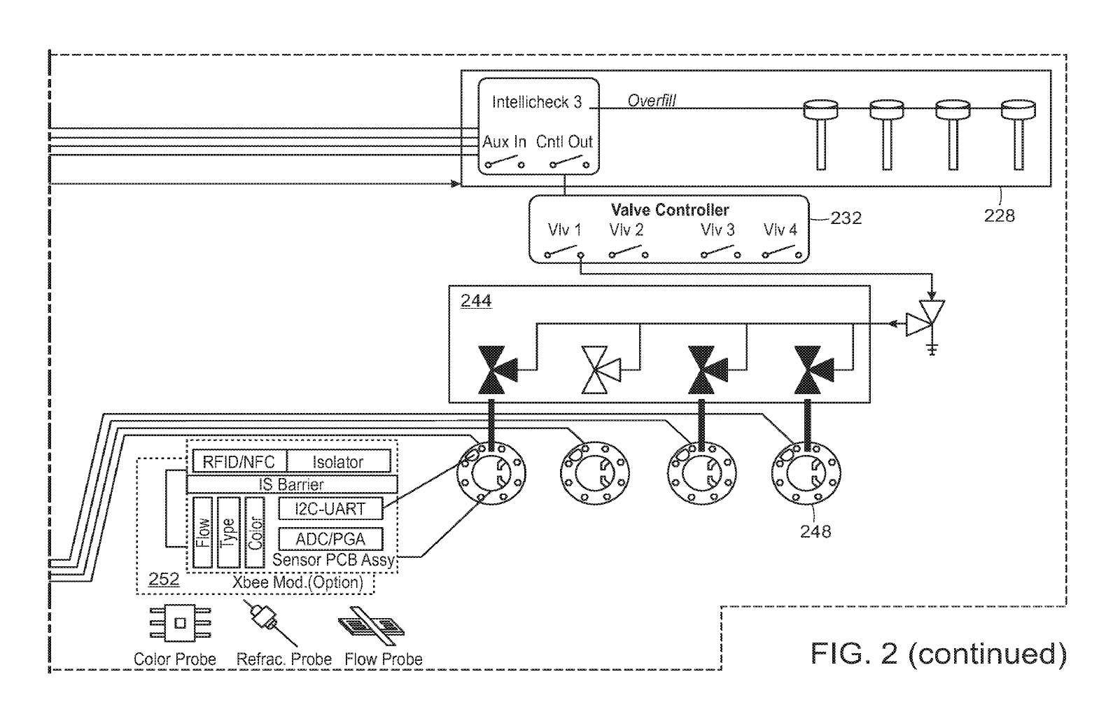

As shown in FIG. 2, a truck 200 and a trailer 204 are provided with interconnected components to facilitate the correct loading/unloading of product. The truck includes known Truck OBC (On-Board Computer) Fleet telematics 208 and a Trailer-to-Truck Connect Assembly (TTCA) 212. The TTCA 212 and the Fleet telematics 208 are powered by a power bus 216.

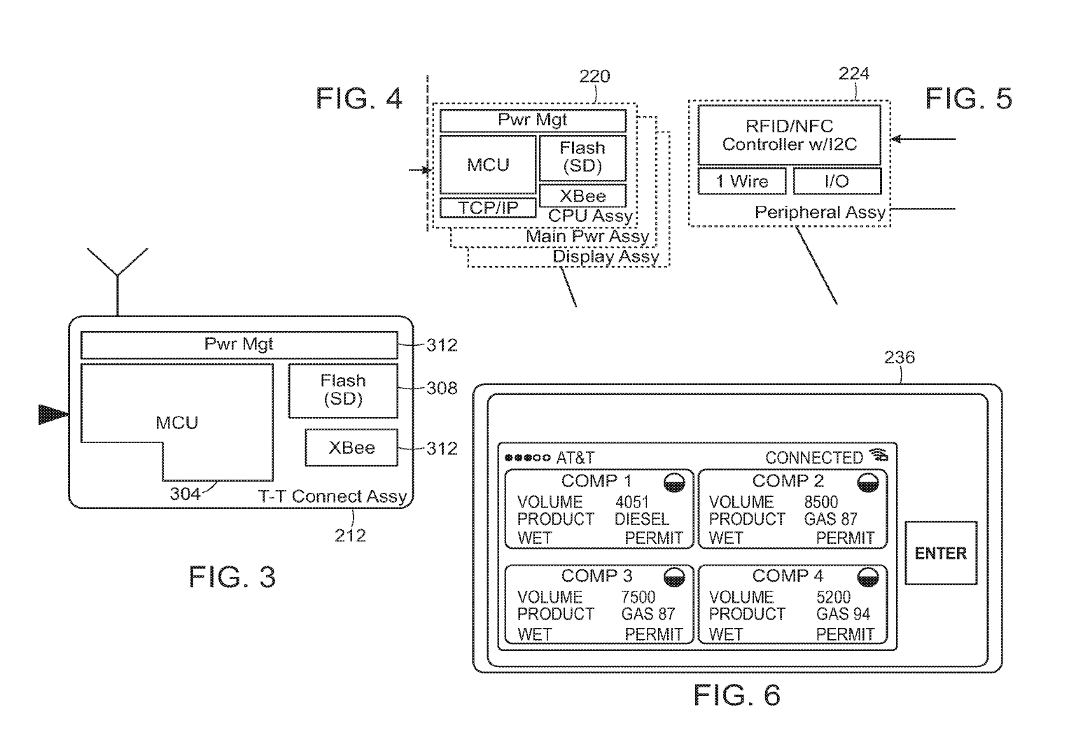

As shown in FIG. 3, the TTCA 212 includes an MCU 304, a Flash memory 308 and an XBee communications module 312 coupled by a power management bus 312 and all in communication with one another.

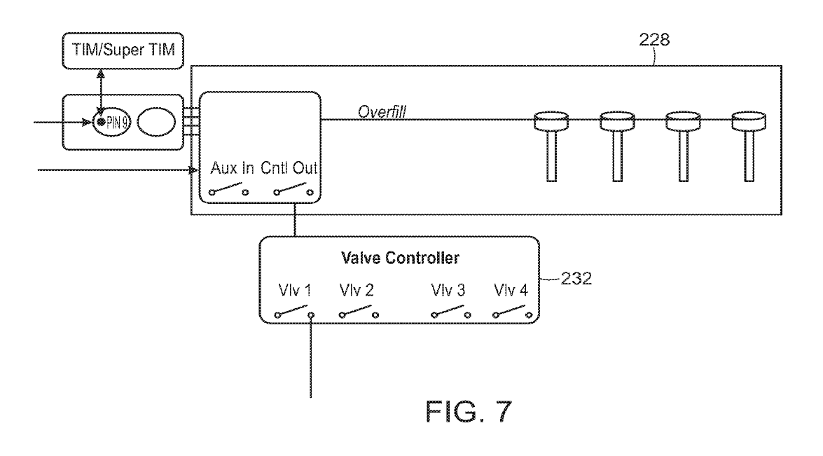

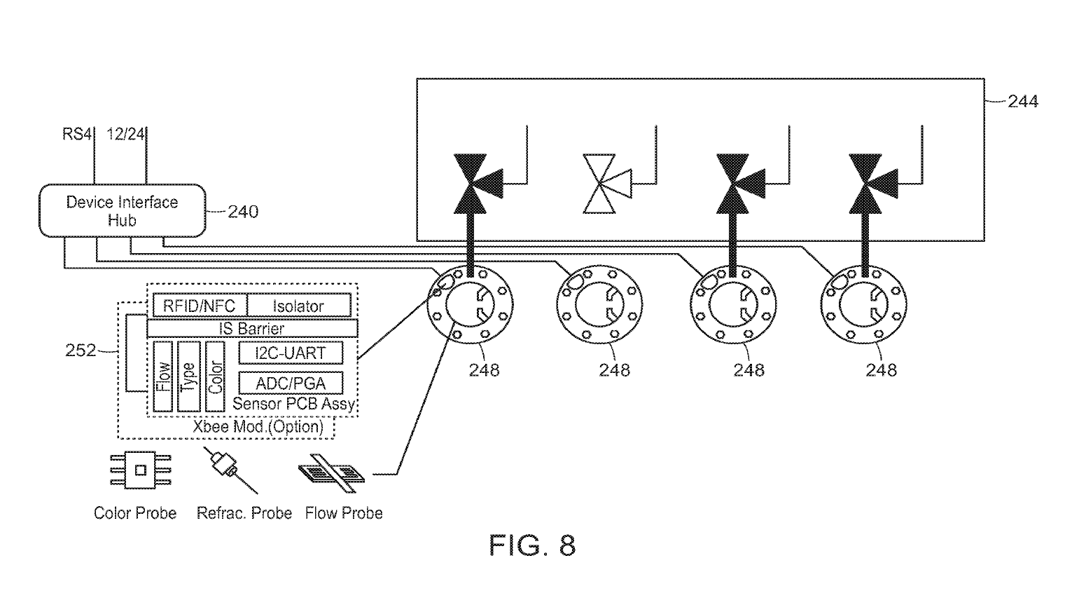

The trailer 204 includes a power management controller (PMC) 220 (see FIG. 4), an overfill detection system 228 (See FIG. 7) coupled to a peripheral controller 224 (see FIG. 5). The overfill detection system 228 may be of a known type available from Scully Signal Company, Wilmington, Mass. A valve controller 232 (see FIG. 7) is coupled to the overfill detection system 228 to control a set of belly valves 244. Each belly valve 244 has a corresponding probe flange 248 coupled through a device hub interface 240 to a product transfer verification system (PTVS) module 236, as shown in FIGS. 6 and 8. The peripheral controller 224 communicatively couples the overfill detection system 228 to the PTVS module 236. In addition, the PMC 220 is coupled to the PTVS module 236.

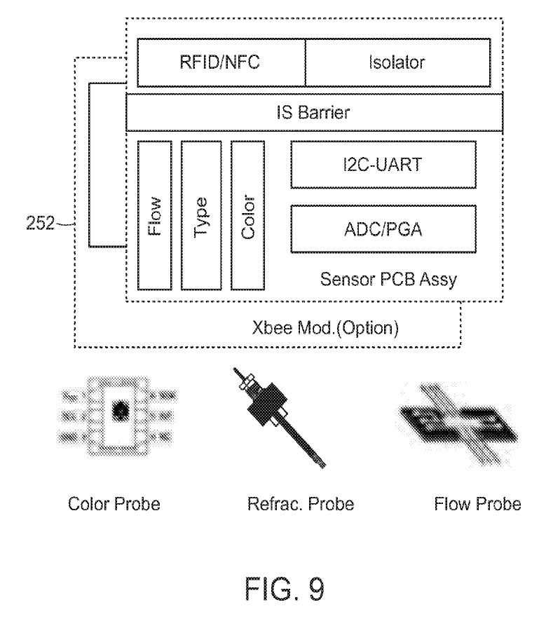

Each probe flange 248 includes a respective sensor PCB assembly 252, FIG. 9, that is coupled to a respective sensor located in the probe flange 248. The probe flange 248 may have one or more probes to detect a type of fluid being passed through based on, for example, its color, its refractive index, its octane value, etc. In addition, a probe measuring flow rate may also be provided in the flange 248.

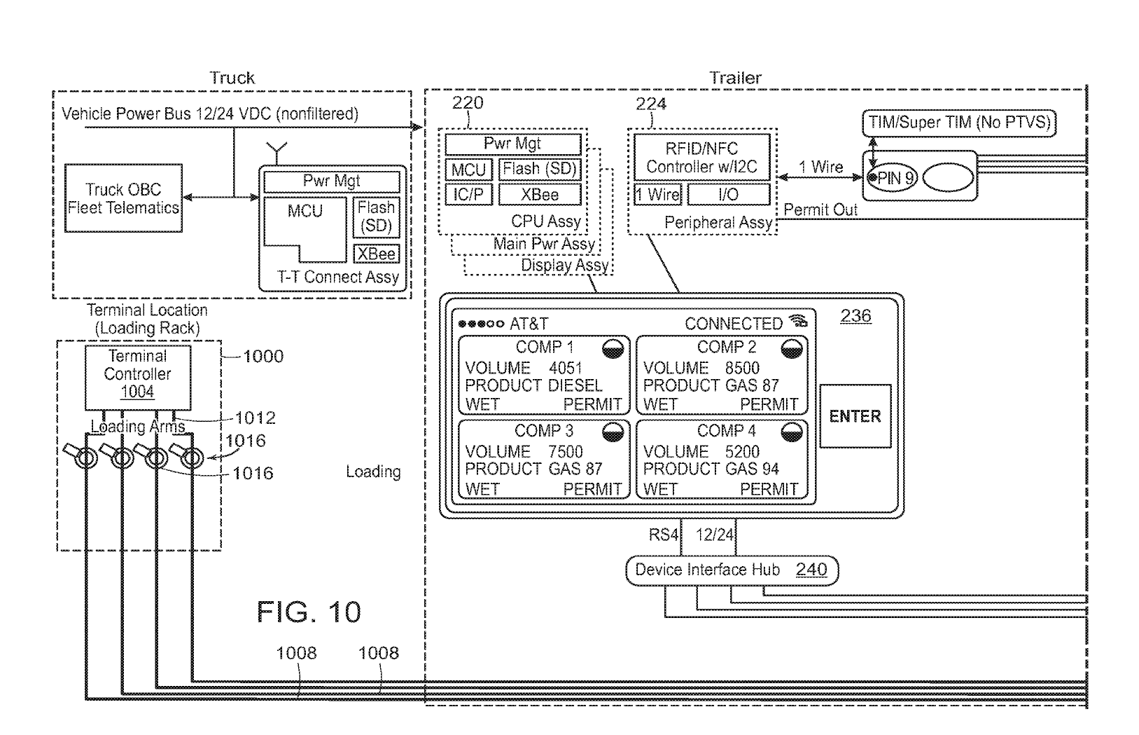

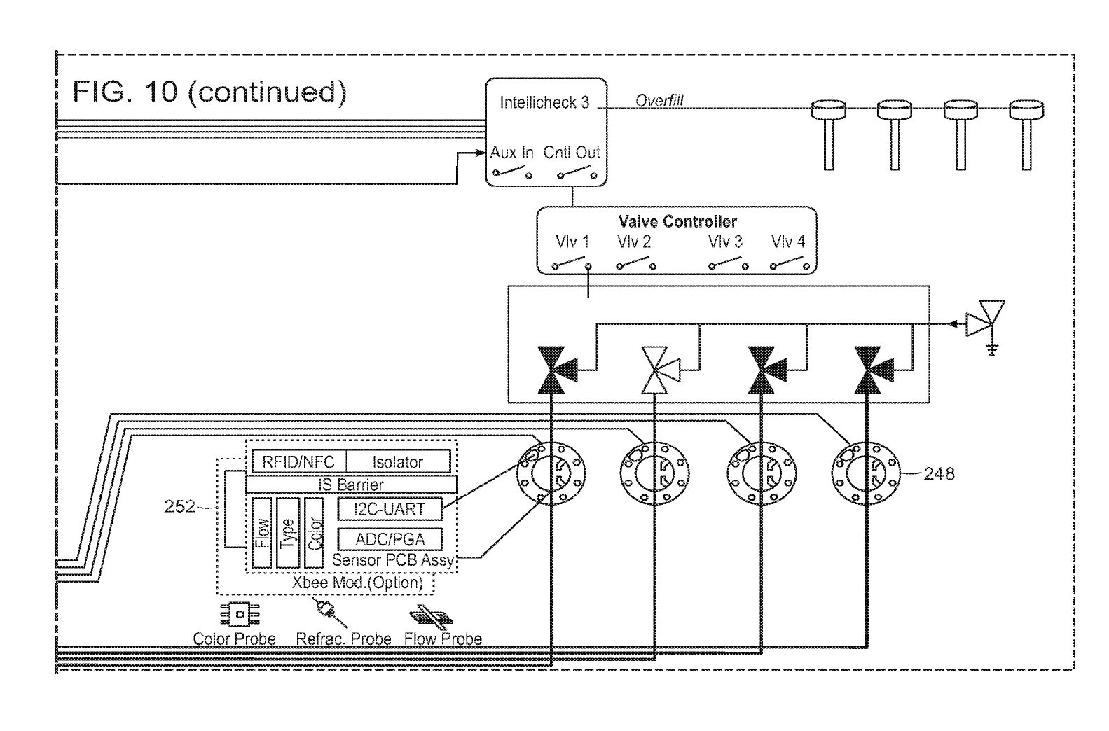

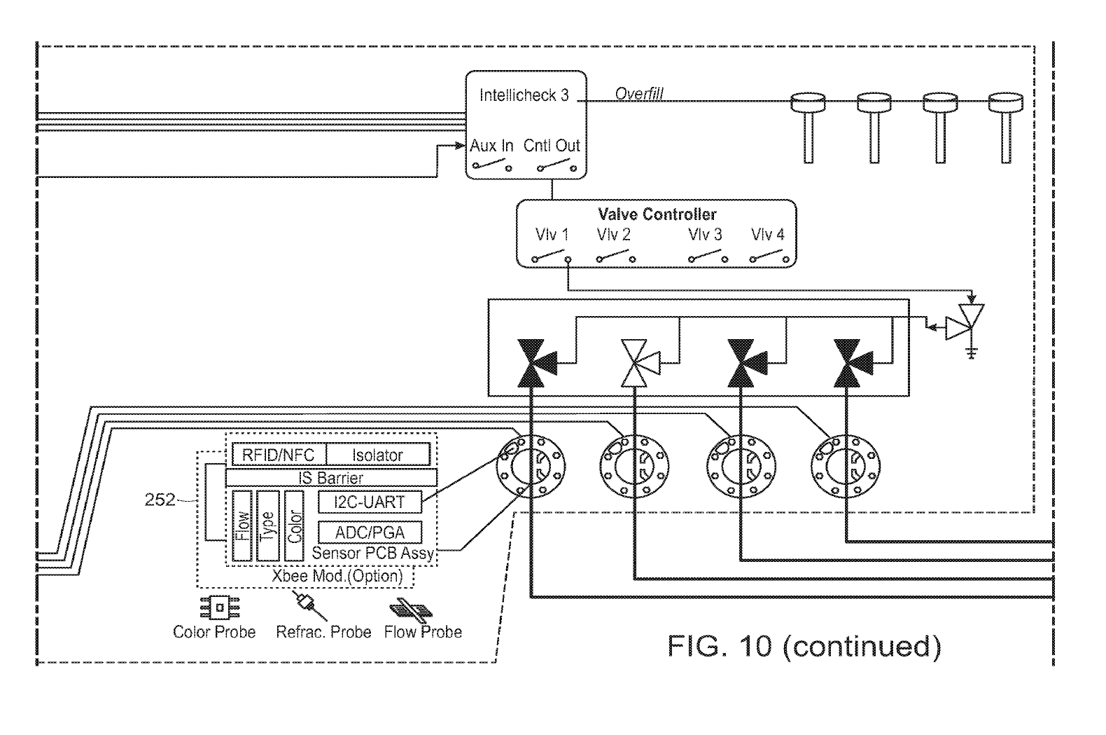

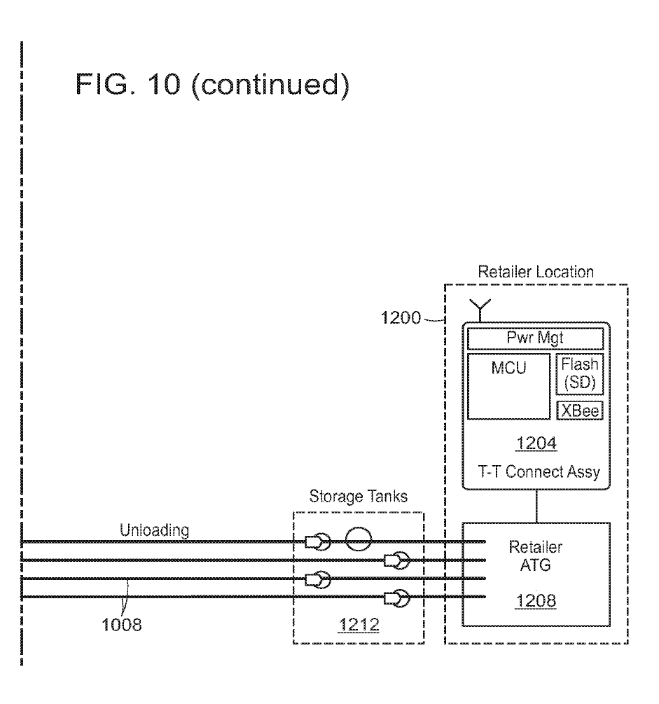

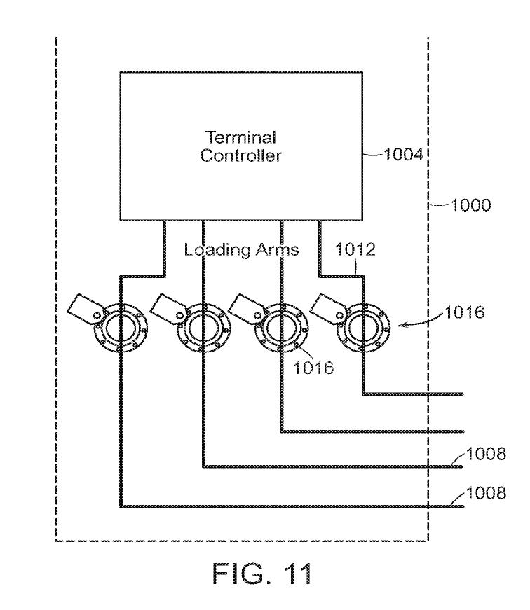

Referring to FIGS. 10 and 11, when loading product into the tanks of the trailer from a terminal 1000 under control of a terminal controller 1004, a plurality of hoses 1008 will be coupled from respective storage tanks to respective tanker compartments. At the terminal, each loading arm 1012 will have a respective tag detector 1016 that confirms or records the type of product that is being transferred.

In operation, the type of product being transferred from the loading rack to a respective compartment on the trailer should be the type that is expected, i.e., not going to contaminate the product that may already be in the compartment, an amount to be transferred that will fit without causing an overfill and/or spill condition and it should be the amount ordered.

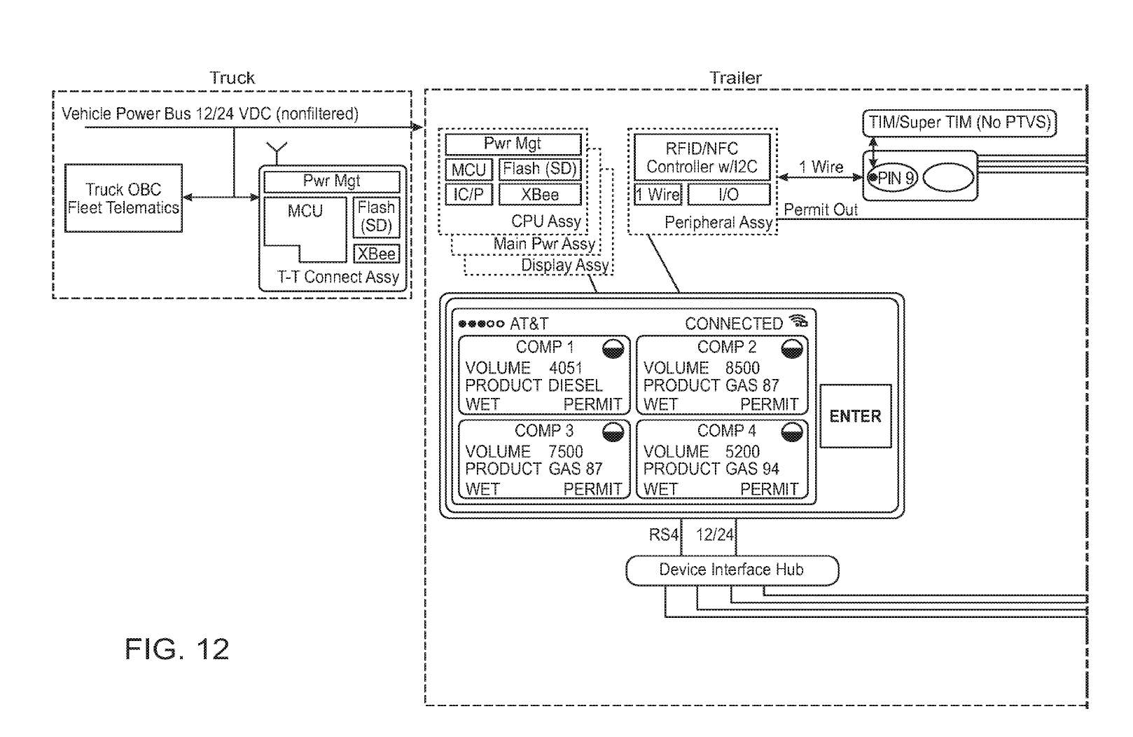

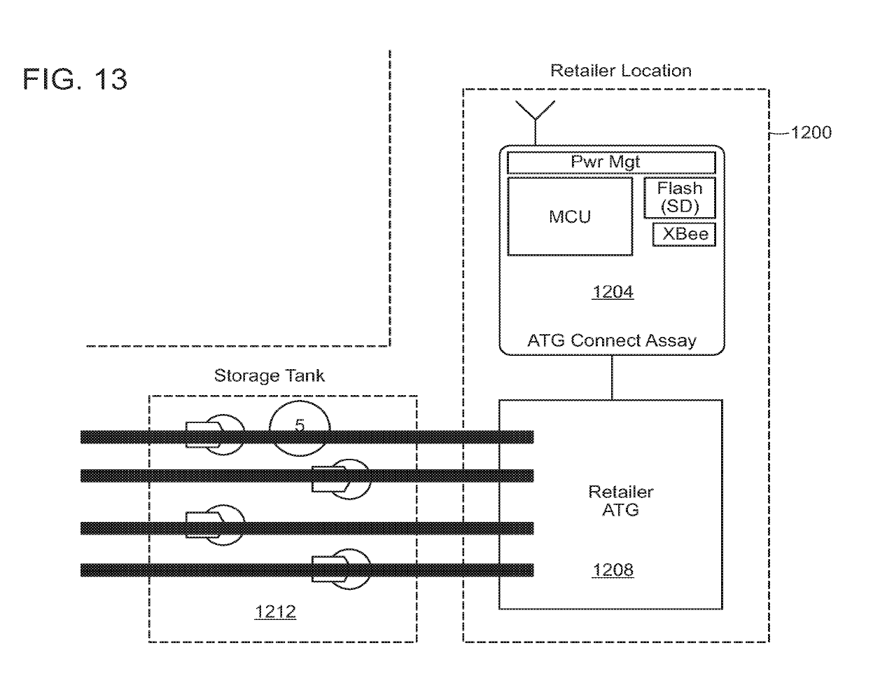

Referring now to FIGS. 12 and 13, when the truck 200 arrives at the delivery destination, for example, a retailer location 1200, then hoses 1008 are connected from the belly valves of the compartments to the storage tanks 1212 of the retailer. Each storage tank has a corresponding tag detector 1016 to confirm that the correct fuel type is being unloaded into the proper tank 1212. The detector TAGs 1016 are connected to a retailer ATG system 1208 coupled to an ATG Connect Assembly 1204 operating to confirm correct unloading processes.

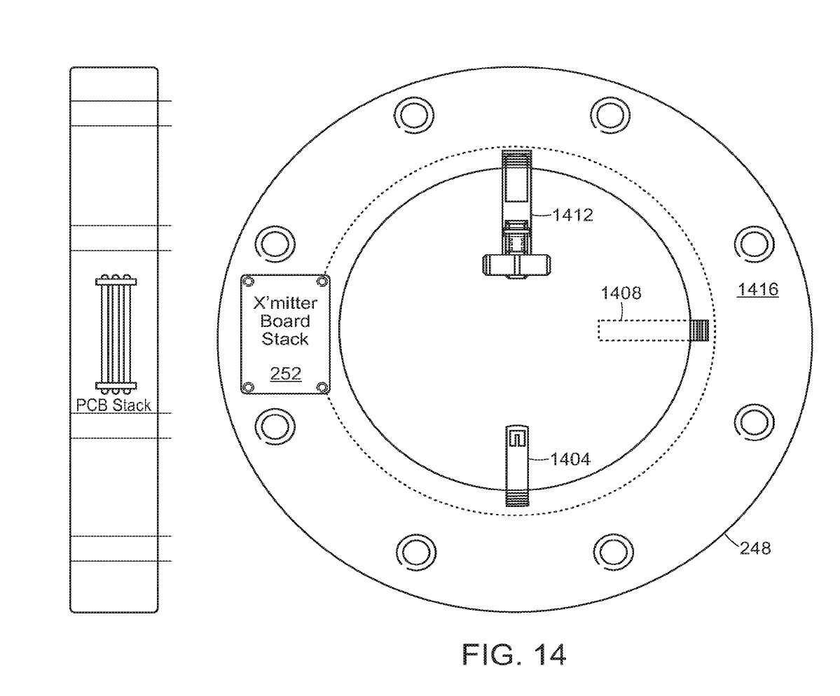

As discussed above, each belly valve 244 has a corresponding probe flange 248 as shown in FIG. 14. The flange 248 includes a frame that may be of any shape, e.g., rectangular or circular, and includes an opening, usually circular in cross-section, and structured as is known in the art to allow for the passage of a fluid. The flange 248 includes a number of probes, i.e., sensors, extending into the area through which the product flows. These probes may include, for example, a flow probe 1404, an octane probe 1408 and a color probe 1412 to sense, respectively, a flow rate, an octane value and a color, of fluid passing through the flange 248. Of course, other types of probes or sensors including, but not limited to, temperature, viscosity, radiation, etc., could be used. In one example, the octane probe 1408 may be implemented as a refractory probe that measures the refractive index of the fluid to determine if it corresponds to an expected octane value.

Each probe is coupled to a signal bus 1416 that runs within the flange and couples to the transmitter board stack or module 252. In one implementation, the signal bus may have power and data lines or power may be on a bus separately provided from the data depending upon the application. The transmitter board 252 couples the information from the probes to the device interface hub 240 (see FIG. 8), via the signal bus 1416, for further processing by the product transfer verification system (PTVS) module 236. The flange 248 may have an external port to receive power and/or transmit or receive data signals. Alternatively, the flange 248 may have its own power source such as a rechargeable battery or similar mechanism.

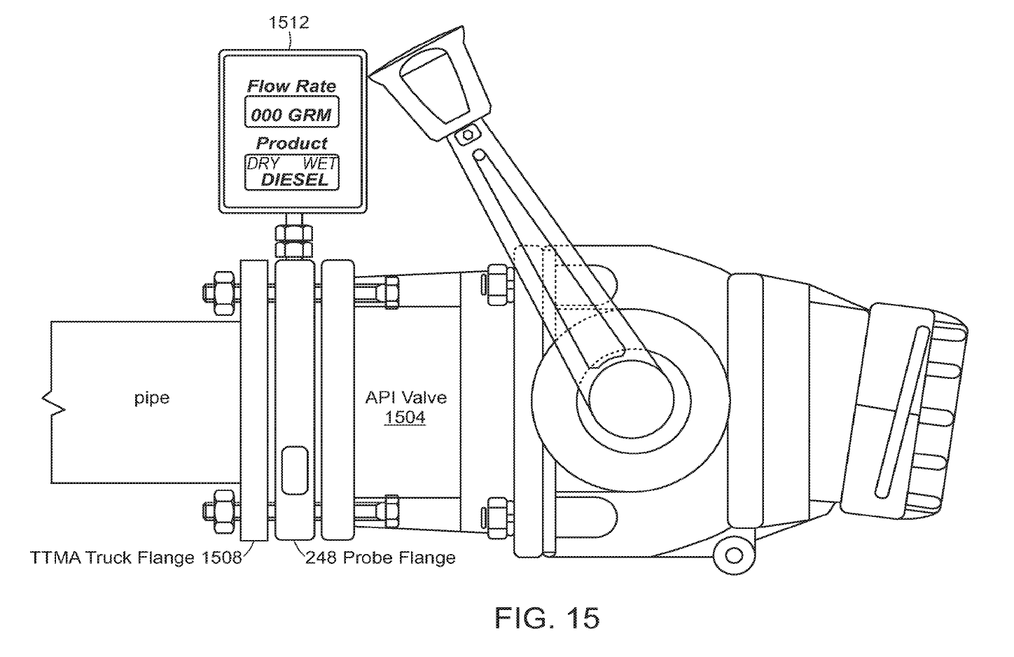

The probe flange 248 may be placed “in-line” between an API valve 1504 and a TTMA Truck Flange 1508 as shown in FIG. 15 to allow product to flow through it. In addition, the flange 248 may have an external display 1512 coupled to the transmitter board stack 252 to display real-time information about the product in the conduit. Further, the transmitter board stack 252 may be configured to wirelessly communicate information from the one or more probes by operation of any known protocol including, but not limited to, Bluetooth and Wi-Fi, for example.

Various implementations of the above-described systems and methods may be provided in digital electronic circuitry, in computer hardware, firmware, and/or software. The implementation can be as a computer program product, i.e., a computer program tangibly embodied in an information carrier. The implementation can, for example, be in a machine-readable storage device for execution by, or to control the operation of, data processing apparatus. The implementation can, for example, be a programmable processor, a computer, and/or multiple computers.

While some of the above-described implementations may generally depict a computer implemented system employing at least one processor executing program steps out of at least one memory to obtain the functions herein described, it should be recognized that the presently described methods may be implemented via the use of software, firmware or alternatively, implemented as a dedicated hardware solution such as in an application specific integrated circuit (ASIC) or via any other custom hardware implementation.

It is to be understood that various aspects of the present disclosure have been described using non-limiting detailed descriptions of implementations thereof that are provided by way of example only and are not intended to be limiting. Features and/or steps described with respect to one implementations may be used with others and not all have all of the features and/or steps shown in a particular figure or described with respect to one of the implementations. Variations will occur to persons of skill in the art.

It should be noted that some of the above described implementations include structure, acts or details of structures and acts that may not be essential and which are described as examples. Structure and/or acts described herein are replaceable by equivalents that perform the same function, even if the structure or acts are different, as known in the art, e.g., the use of multiple dedicated devices to carry out at least some of the functions described as being carried out by the processor.

1. A coupling flange, comprising:
a coupling frame having an opening defining a fluid path configured to be fluidly coupled with a fluid source;
at least one sensor having a sensing portion configured to measure at least one characteristic of a fluid, the sensor provided in the coupling frame such that the sensing portion is located in the fluid path;
a signal bus, provided in the coupling frame, coupled to the at least one sensor; and
a transmitter module, coupled to the signal bus, configured to transmit sensor data received from the at least one sensor.

2. The flange of claim 1, wherein the signal bus is encapsulated within the frame.

3. The flange of claim 1, wherein the transmitter module is encapsulated within the frame.

4. The flange of claim 1, wherein each of the sensor and the transmitter module is configured to receive power via the signal bus.

5. The flange of claims 1, wherein the transmitter module is configured to wirelessly transmit the received sensor data.

6. The flange of claim 1, wherein the sensing portion measures at least one of:
a color, a temperature, an octane value, a viscosity, a refraction index and a radiation level.

7. The flange of claim 1, further comprising:
a display module mounted on the frame and coupled to the signal bus,
wherein the display module is configured to visually display information determined as a function of the at least one measured characteristic received from the sensor.

8. The flange of claim 1, wherein the sensing portion is further configured to contact a fluid flowing in the defined opening.

9. The flange of claim 1, wherein the sensing portion is further configured to withstand flowing or standing fluid in the fluid path.

10. A method of controlling a pending transfer of a fluid product to a receiving compartment, the method comprising:
measuring at least one characteristic of the pending fluid product;
identifying, as a function of the at least one measured characteristic, a type of the pending fluid product;
determining if the identified fluid type corresponds to the receiving compartment; and
preventing the pending transfer if the identified fluid type does not correspond to the receiving compartment.

11. The method of claim 10, wherein measuring at least one characteristic of the pending fluid product comprises:
providing a coupling flange in-line with a fluid path conduit, the coupling flange including a sensor probe; and
contacting, with the sensor probe, the fluid product in the fluid path conduit.

12. The method of claim 11, wherein the sensor probe is configured to measure at least one of: color, temperature, octane value, viscosity, refractive index and radiation level.

13. The method of claim 10, wherein measuring at least one characteristic of the pending fluid product is performed while the fluid product is not flowing in the fluid path conduit.

14. The method of claim 10, wherein preventing the pending transfer comprises diverting the fluid product to an alternate compartment different from the receiving compartment.

15. The flange of claim 6, further comprising:
a display module mounted on the frame and coupled to the signal bus,
wherein the display module is configured to visually display information determined as a function of the at least one measured characteristic received from the sensor.

16. The method of claim 12, further comprising:
receiving at least one measured characteristic from the sensor probe;
determining information as a function of the received at least one measured characteristic; and
visually displaying the determined information on a display module mounted on the coupling flange.
